(19) United States
(12) Patent Application Publication
(10) Pub. No.: US 2007/0190101 A1
(43) Pub. Date: Aug. 16, 2007

(54) Flowable bone grafts

(76) Inventors: Chunlin Yang, Belle Mead, NJ (US); Jacky Au-Yeung, San Francisco, CA (US); Iksoo Chun, Princeton, NJ (US); Mark Davis, San Jose, CA (US); Lu Liu, San Mateo, CA (US); Aruna Nathan, Bridgewater, NJ (US); Vivek Shenoy, Sunnyvale, CA (US); Mark Timmer, Jersey City, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/815,000

(22) Filed: Mar. 31, 2004

(51) Int. Cl. A61K 35/32; A61K 38/17; A61K 38/18

(52) U.S. Cl. 424/423; 424/602; 514/12; 424/93.7

(57) ABSTRACT

The present invention is directed to bone graft compositions suitable for administration to the body via a cannula, where the compositions contain mineralized collagen particles and a fluid biocompatible carrier having the mineralized collagen particles substantially uniformly distributed there through, which particles contain bound mineralized collagen fibrils substantially uniformly distributed there through and a binder for the fibrils; and to methods of making such particles.

### FIELD OF THE INVENTION

The present invention relates to particles containing bound mineralized collagen fibrils and flowable bone graft compositions utilizing such particles.

### BACKGROUND OF THE INVENTION

The regenerating potential of human bone appears to be limited. Bone graft has been employed for repairing discontinuity defects in bone that can result from traumatic injuries, congenital deformities, and tumor resection. Bone graft also has been used in bone contouring and augmentation, as well as in stimulating formation of bone at specific sites within the body, e.g. a spinal fusion.

The clinical approach to repairing or restoring bone involves substituting the missing tissue with an autogeneic and allogeneic bone graft or processed bone. Problems associated with autogeneic bone grafting include a limited source of donor bone and the need for an additional surgery to procure the tissue, which engenders the risk of high morbidity at the donor site. For allogeneic bone grafts, potential risks include the transfer of diseases, immunological reactions from the host, poor osteogenic capacity of the transplanted bone, and high cost associated with a bone banking system.

Another approach used is a conformational method whereby an implant, usually composed of metal, ceramic, or other inorganic material in a structured form intended to mimic the shape of the missing bone, is inserted into the site in which bone replacement is required. There is a risk that the host will reject the material or that the implant will fail to integrate with normal skeletal tissue. Ceramic materials such as tricalcium phosphate, although biocompatible with the host and bone, appear to lack sufficient mechanical properties of bone for general utility when used as an implant and the bone does not consistently grow into and become incorporated within the implant.

A third method involves the process known as osteoinduction, which occurs when a material induces the growth of new bone. Three approaches for inducing new bone tissue have been reported in the literature: 1) implantation of cytokines such as BMPs in combination with appropriate delivery systems that will lead to new healthy bone formation at the target site; 2) transduction of genes encoding cytokines with osteogenic capacity to cells at the repair site; and 3) transplantation of osteogenic cells. However, such osteoinductive material must be delivered to the desired site in an appropriate graft matrix.

Ideal characteristics for a grafting matrix include spatial and compositional properties that will attract and guide the activity of respective cells. The regeneration of lost or damaged tissue requires that reparative cells adhere, migrate, grow, and differentiate in a manner that results in the synthesis of proper new tissue.

The use of mineralized collagen fibers has been reported for use in bone repair. U.S. Pat. No. 5,231,169 by Constantz et al discloses mineralized collagen fibers prepared by forming calcium phosphate mineral in situ in the presence of dispersed collagen fibrils. The fibrils may be further treated and/or combined with other materials such as hydroxyapatite or osteoinductive materials and used for treating bone disorders. U.S. Pat. No. 5,532,217 by Silver et al discloses a process for mineralization of collagen fibers prepared by extruding a collagen solution into a fiber-forming buffer. The fibers may be admixed with physiologically acceptable inert carriers to form ointments, gels, gel creams or creams. U.S. Pat. No. 6,187,047 B1 by Kwan et al discloses a porous, three-dimensional bone graft matrix formed from mineralized collagen fibrils.

While the art has disclosed the use of mineralized collagen fibers as noted above, it has not disclosed or suggested flowable bone graft compositions that may be administered in a flowable form to the body via a cannula of a medical device, e.g. a needle, in which case materials and compositions noted above would not be conducive for such use. The present invention provides particles containing bound mineralized collagen fibrils and flowable bone graft compositions utilizing such particles that are able to fill and to be densely packed within irregular-shaped bone defects and cavities while providing compositional characteristics similar to bone extracellular matrix. Furthermore, the flowability of compositions of the present invention facilitates the use of bone grafts in non-invasive and minimally invasive surgical procedures.

### SUMMARY OF THE INVENTION

The present invention is directed to a bone graft composition suitable for administration to the body via a cannula, where the compositions contain mineralized collagen particles and a fluid biocompatible carrier comprising the mineralized collagen particles substantially uniformly distributed there through, which particles comprise bound mineralized collagen fibrils substantially uniformly distributed there through and a binder for said fibrils; and to methods of making such particles.

### BRIEF DESCRIPTION OF THE FIGURES

FIG. 1 shows a scanning electron micrograph (SEM) of rotor-milled mineralized collagen particles containing bound mineralized collagen fibrils.

FIG. 2 shows a scanning electron micrograph (SEM) of cryo-milled mineralized collagen fibrils.

FIG. 3 shows a scanning electron micrograph (SEM) of particles containing bound mineralized collagen fibrils.

FIG. 4 shows a micrograph of particles containing bound mineralized collagen fibrils.

### DETAILED DESCRIPTION OF THE INVENTION

As used herein, mineralized collagen fibrils comprise collagen fibrils having a substantially uniform distribution of calcium phosphate crystals distributed there through, as further described herein below. The fibrils used to prepare particles of the present invention may have a diameter of from less than one micron up to about 200 microns, preferably from about 5 to about 50 microns. The length of such fibrils may range from about 10 microns up to about 3 millimeters, preferably from about 100 microns to about 1 millimeter. In certain embodiments it is even more preferred that the average length is less than about 300 microns.

The collagen to be mineralized may come from mineralized sources, e.g. hard tissue such as bone, or unmineralized sources, e.g. soft tissue such as tendon and skin, although unmineralized collagen sources usually are used. Preferably, the collagen includes a combination of three strands of α-collagen chains. The collagen may be from a young source, e.g. calf, or a mature source, e.g. cow of 2 or more years. The particular source of the collagen may be any convenient animal source, mammalian or avian, and may include bovine, porcine, equine, chicken, turkey, or other domestic source of collagen, including recombinant collagen.

One method of producing the mineralized collagen fibrils utilized in particles and compositions of the present invention is described in U.S. Pat. No. 5,231,169 (Constantz), the content of which is hereby incorporated by reference as if set forth in its entirety. Other methods of making mineralized collagen fibrils also are know to those skilled in the art. As used herein, calcium phosphate is used to denote those materials belonging to the general class of phosphate salts as is know to those skilled in the art of bone substitutes, including, without limitation, calcium hydroxyapatite, calcium hydroxy/fluorapatite, brushite, dahlite, monetite, phosphated calcium carbonate (calcite), oxtacalcium phosphate, or tricalcium phosphate, where the choice of stoichiometry of the calcium and the phosphate, as well as the presence of other ions, will result in the particular composition. The calcium phosphate is formed in situ in a dispersion of collagen fibrils by the simultaneous gradual addition, preferably continuous addition, of a source of soluble calcium and a source of soluble phosphate. Besides a source of calcium and phosphate, sources of other ions may be employed, such as carbonate, chloride, fluoride, sodium, or ammonium.

The mineral phase of the mineralized collagen fibrils will usually have a Ca:P stoichiometric ratio of from about 1.2:1 to about 1.8:1, hexagonal symmetry and preferably be a member of the hydroxyapatite mineral group. The weight ratio of the collagen fibrils to calcium phosphate mineral generally will be in the range of from about 9:1 to about 1:1, preferably about 7:3. The amount of collagen present in the mineralized collagen fibrils generally will be from about 80% to 30% based on the total weight of the fibrils. The mineralized collagen may be cross-linked using a variety of cross-linking agents, such as formaldehyde, glutaraldehyde, chromium salts, di-isocyanates or the like.

In one aspect of the invention, particles containing bound mineralized collagen fibrils substantially uniformly distributed there through are prepared. Agglomerates of the fibrils are bound in such a way that the particles possess mechanical integrity necessary for combining with a flowable carrier medium for the particles, thus forming a flowable bone graft composition, and subsequent administration of the composition to the body. The term flowable is used herein to denote that physical state where the compositions will flow upon application of forced required to administer such compositions through a cannula of a medical device as described herein below, yet will remain substantially immobile after administration to a contained site in the body to be treated, thereby providing continued treatment to the site.

Particles of the present invention must be of appropriate size so as to be useful in flowable bone graft compositions of the present invention. If the mineralized collagen particles are too small, the particles may be difficult to disperse in the bone graft compositions of the present invention. If the particles are too large, the particles may be difficult to administer in the form of a flowable composition. In certain embodiments of the invention particles of the present invention will have an aspect ratio of from about 100:1 to 1:1; in other embodiments from about 50:1 to 1:1; and in yet other embodiments from about 30:1 to 1:1. Depending on the contemplated method of administration to the body and bone disorder to be treated, the average diameter of the mineralized collagen particles may range from about 10 microns up to about 5 millimeters.

Where the compositions are to be administered by injection via a relatively small diameter cannula, e.g. a 14-gauge or 16-gauge needle, the particles are a size effective to pass through the needle and also to prevent the particles from settling-out or phase separating from the carrier medium in the bone graft compositions prior to or during administration. In these cases, the aspect ratio of the particles preferably will range from about 30:1 to 1:1, and the average particle diameter may range from about 10 microns to about 1,000 microns, more preferably less than about 500 microns, and even more preferably the aspect ratio will be less than about 5:1 and the average diameter less than about 250 microns.

In cases where administration is to be via a larger diameter cannula, phase separation may not be an issue, in which case the average diameter of the particles may range from about 250 microns to about 5 millimeters. In other such embodiments the average diameter of the particles may range from about 500 microns to about 3 millimeters, or from about 1 to 2 millimeters. Once having the benefit of the disclosure herein, one skilled in the art will be able to readily ascertain the appropriate particle size for the composition, method of administration and treatment contemplated.

Depending on the process used to prepare particles of the present invention, the particles may be irregularly shaped agglomerates of bound fibrils, or may be of a more spherical configuration. The particles may comprise a substantially solid structure, or may comprise a porous structure, which renders the particles compressible to some degree. Such compressibility may aid during administration of the particles. Porous particles also may have the ability to absorb the liquid carrier, typically containing a bioactive material, which may provide additional benefit once administered to the body.

In one embodiment for making particles of the present invention, a porous, three-dimensional matrix may be prepared by combining the mineralized collagen fibrils described above with a binder component, preparing a foam or sponge containing the fibrils dispersed throughout the binder, and then cross-linking with the cross-linking agents mentioned above. Preferably a proportion of about 10% (wt:wt) binder is used. One method of forming a porous, three-dimensional mineralized collagen fibrous matrix is described in U.S. Pat. No. 6,187,047, the content of which is incorporated herein as if set forth in its entirety. The preferred binder for forming the matrix is soluble collagen, although other binders that may be used include, without limitation, gelatin, polylactic acid, polyglycolic acid, copolymers of lactic and glycolic acid, polycaprolactone, carboxymethylcellulose, cellulose esters (such as the methyl and ethyl esters), cellulose acetate, dextrose, dextran, chitosan, hyaluronic acid, ficol, chondroitin sulfate, polyvinyl alcohol, polyacrylic acid, polypropylene glycol, polyethylene glycol, poly(vinyl pyrrolidone), alginic acid and water-soluble methacrylate or acrylate polymers.

Particles of the present invention containing mineralized collagen fibrils may then be formed from the sponge or foam sheets described immediately above by mechanical means with equipment such as shredders, rotary cutters and dicers, pulvarizers, peripheral speed mills and fluid energy superline mills. The process parameters are selected so as not to disassociate the calcium phosphate mineral component from the collagen fibrils. A preferred method of forming the mineralized collagen particles by this method is by cryo-milling, whereby the sponge matrix containing the bound mineralized collagen fibrils is frozen with liquid nitrogen and then pulverized. Another preferred method is by rotor milling, whereby the mineralized collagen sponge can be processed at room temperature and the shearing action between the rotor and the stationary blade maintains a fibrillar, irregularly shaped structure for the particles. The particles are then segregated, e.g. by sieves or other methods of selection, to obtain a desired particle size distribution, again depending on the particular composition, use and method of administration being contemplated.

In other embodiments for preparing mineralized collagen particles, intact mineralized collagen fibrils as described in Constantz may be incorporated with a binder solution. As used herein, the term “intact” is intended to denote fibrils that, once formed, do not undergo micronization. An emulsion comprising the binder solution is formed, followed by crosslinking, as described below, thus forming the particle containing bound mineralized collagen fibrils. Aqueous solutions typically are used as the binder component, with an oil phase, e.g. olive oil, used as the other emulsion component. Solvents other than olive oil may be used in the process provided that the solvent is immiscible in water and the crosslinker is soluble in the solvent. Typical crosslinking agents include, without limitation, glutaraldehyde, diisocyanates, formaldehyde, carbodiimides, e.g. 1-ethyl-3-(3-dimethylaminopropyl) carbodiimide, and glyceraldehydes. The fibrils described in Constantz alternatively may be micronized, i.e. cut or otherwise processed, to reduce the length of the fibril prior to preparation of the particles in this manner where smaller diameter particles are preferred. Binding of the fibrils in this way provides structural integrity to the particle, increases the bulk of the particle and provides a more regularly shaped, spherical particle. The particles may be sterilized by standard sterilization techniques (gamma irradiation, e-beam, ethylene oxide etc.).

Such particles are suitable for use in flowable bone graft compositions of the present invention. The binding material may be of any material suitable for such use, although soluble collagen, either denatured or native, is preferred.

In one process for making the particles, mineralized collagen fibrils, whether in the form of intact fibrils or micronized fibrils, may be dispersed and suspended in an aqueous solution of water-soluble, denatured collagen that has been prepared by heating a solution of native collagen to around 60° C.-80° C., as described in Example 3 herein below. The aqueous slurry then is poured into an oil phase, e.g. an olive oil bath that is maintained at about 40° C. and agitated to form a substantially homogeneous emulsion. The emulsion is transferred to an ice bath with continued agitation so that the denatured collagen forms a gel coating around the mineralized collagen fibrils. A water-miscible solvent is added to the suspension of gelatin-coated agglomerates that are then separated by filtration and crosslinked with any of the suitable crosslinking agents mentioned above to form the particles. Mineralized collagen particles thus formed are washed with, e.g. acetone, and isolated.

In another process, particles containing the intact mineralized collagen fibrils or milled mineralized collagen fibrils may be prepared using native collagen or denatured collagen, as the binder material. The mineralized collagen fibrils are mixed with an aqueous solution of water-soluble collagen and the pH of the mixture adjusted to between about 9 and about 13 until a homogeneous aqueous slurry is formed. The aqueous slurry is then dispensed drop-wise into a stirred oil phase, e.g. a bath of olive oil, maintained at 10° C. to 30° C. After the water-in-oil emulsion is formed, the particles are stabilized by the addition of a surfactant such as Span 85 (Sigma, Inc.) Other surfactants like Sorbitan tristearate (Span 65), Sorbitan sesquioleate (Arlacel 83), Glyceryl monostearate, Tergitol 15-S-3, Tergital 15-S-5, sorbitan monooleate (Span 80), Sorbitan monostearate (Span 60) etc. could also be used. The stabilized particles are then crosslinked by the addition of a crosslinker. When soluble collagen or denatured collagen (gelatin) is used as the binder, concentrated glutaraldehyde can be used as the crosslinker. The crosslinked particles are then separated from the water-oil emulsion by the addition of excess water. Due to their higher density, the particles sink to the bottom of the aqueous phase and are separated from the oil easily. The addition of excess water serves two purposes; a) quenching the crosslinking reaction, and b) removing the residual crosslinker, surfactant and oil.

Once isolated from the respective emulsions, the particles may be dried by various means, although vacuum drying is preferred. Vacuum drying may be conducted either at room temperature, or in certain embodiments the isolated particles may be lyophilized, in which case the particles are first frozen, thus trapping frozen solvent within the particle structure, and then the frozen solvent is removed under vacuum, thus providing porosity in the particle where the frozen solvent has been removed. Particles dried via lyophilization, and thus having such a porous structure, may provide additional advantages when used in compositions for certain uses, due in part to the physical compressibility imparted to the particle and to the presence of pores that may be able to absorb materials such as a carrier of bioactive agents.

Another method for binding the mineralized collagen agglomerates is by spray drying, whereby an aqueous solution of soluble collagen, native or denatured, is sprayed onto the mineralized collagen agglomerates and then evaporated by drying. To avoid denaturation of collagen fibril in the thus coated mineralized collagen particles, the process temperature during spray drying should be maintained below 60° C., preferably around 40° C.

Flowable bone graft compositions are prepared utilizing the mineralized collagen particles of the present invention. In one embodiment of the invention, the compositions comprise mineralized collagen particles of the present invention substantially uniformly distributed through an inert, biocompatible, liquid carrier for the particles. In other embodiments, the compositions further comprise a bioactive material, also substantially uniformly distributed through the carrier. In other embodiments, the composition may comprise the mineralized collagen particles substantially uniformly distributed through a bioactive material that serves as the carrier for the particles as well.

Compositions of the present invention generally comprise from about 1.5 to about 35 weight percent of the mineralized collagen particles. In certain embodiments, the compositions preferably may comprise from about 10 to about 25 weight percent of the particles. The composition may comprise from about 98 to about 65 weight percent of the carrier, preferably from about 90 to about 75 percent. In cases where the bioactive agent serves as the carrier, the composition may comprise from about 98 to about 65 weight percent of the material, preferably from about 90 to about 75 percent. Where the compositions comprise both a carrier and a bioactive agent, the ratio of carrier to bioactive agent may range from about 60:40 to about 40:60 (w:w).

In other embodiments of the invention, compositions may comprise from about 1.5 to about 7.5 weight percent of the mineralized collagen particles. Preferably, the compositions may comprise from about 2.0 to about 6.0 weight percent of the particles. More preferably, the compositions may comprise about 2.5 to about 5 weight percent of the particles. The composition may comprise from about 98.5 to about 92.5 weight percent of the carrier, preferably from about 98 to about 94 percent, more preferably from about 97.5 to about 95 percent.

Carriers suitable for use in compositions of the present invention are fluid, biocompatible, biodegradable and pharmaceutically acceptable. The carrier preferably is aqueous-based. Preferred carriers include, without limitation, hyaluronic acid, succinalyted collagen, carboxymethylcellulose (CMC), gelatin, collagen gels, fibrinogen, thrombin, liquid alkyd polyesters, such as monooleoyl glyceride-co-succinate, and liquid polyhydroxy compounds.

The liquid carrier should be fluid enough so as to substantially wet the particles when dispersed therein and to provide flowability to the composition, while having a viscosity effective to provide the bone graft compositions with properties necessary for its contemplated use. In embodiments where the composition must be injectable through a relatively narrow opening, e.g. via a needle, the carrier must be viscous enough to provide for a stable dispersion of the particles in the carrier, yet fluid enough to pass through the needle under forces ordinarily encountered during standard injection of materials into the body, preferably without phase separation of the particles from the carrier medium. In embodiments where the composition is to be delivered via a larger cannula, the compositions must be able to flow through the cannula to the targeted site. The exact properties required, i.e. flowability, viscosity, ability to suspend the particles, etc., will depend on the structure and geometry of the particular cannula through which the composition is to be delivered and on the particular device comprising the cannula.

Examples of devices from which the compositions may be administered include the INSITE™ system (The Bright Group, Inc.). The INSITE system consists of cannulas of diameters 19, 22- and 26 mm and lengths ranging from 40-90 mm.

Compositions of the present invention may further include a bioactive agent. Such bone grafting compositions may be useful in applications such as spinal fusion, filling bone defects, fracture repair, grafting periodontal defects, maxifacial reconstruction and joint reconstruction, as well as in other orthopedic surgical uses. Such agents include, without limitation, osteoinductive materials.

Bioactive agents suitable for use with the present invention include without limitation, cell attachment mediators, such as peptide-containing variations of the “RGD” integrin binding sequence known to affect cellular attachment, biologically active ligands, and substances that enhance or exclude particular varieties of cellular or tissue ingrowth. The bioactive agent may be present in monomeric or dimeric forms and may be peptides or polypeptides with bioactivity similar to morphogenic proteins. Suitable examples of such bioactive agents include integrin binding sequence, ligands, bone morphogenic proteins (in both monomeric and dimeric forms), epidermal growth factor, IGF-I, IGF-II, TGF-β I-III, growth differentiation factor, parathyroid hormone, vascular endothelial growth factor, glycoprotein, lipoprotein, bFGF, TGF-superfamily factors, BMP-2, BMP-4, BMP-6, BMP-12, BMP-14, MP-52, sonic hedgehog, GDF5, GDF6, GDF8, PDGF, small molecules that affect the upregulation of specific growth factors, tenascin-C, fibronectin, thromboelastin, thrombin-derived peptides, heparin-binding domains, and the like. Furthermore, the bone replacement material may comprise mineralized collagen particles mixed with a biologically derived substance selected from the group consisting of demineralized bone matrix (DBM), platelet rich plasma, bone marrow aspirate and bone fragments, all of which may be from autogenic, allogenic, or xenogenic sources.

In certain embodiments of the present invention, the mineralized collagen particles are combined with a bioactive material that also serves as the flowable carrier. One preferred embodiment comprises the mineralized collagen particles dispersed in fresh bone marrow aspirate, whereby the marrow serves both as a carrier and a source of osteogenic growth factors and progenator cells.

In yet other embodiments, compositions of the present invention may further comprise selected cell types, depending on the particular contemplated treatment. Cells that can be seeded or cultured in the mineralized collagen particles of the present invention include, but are not limited to, bone marrow cells, mesenchymal cells, stromal cells, stem cells, embryonic stem cells, osteoblasts, precursor cells derived from adipose tissue, bone marrow derived progenitor cells, peripheral blood progenitor cells, stem cells isolated from adult tissue, and genetically transformed cells, or combinations of the above.

Yet other embodiments of the present invention may comprise further agents such as: chemotactic agents; therapeutic agents (e.g., antibiotics, steroidal and non-steroidal analgesics and anti-inflammatories, anti-rejection agents such as immunosuppressants, and anti-cancer drugs); genes and therapeutic gene agents; and other such substances that have therapeutic value in the orthopaedic field.

Embodiments of the present invention can be readily prepared as needed. In one embodiment, the mineralized collagen particles can be pre-packed within a syringe to which the liquid carrier and bioactive materials can be added and mixed in a closed system. One preferred closed system includes two syringes and a co-joining stopcock, whereby the collagen particles and liquid components initially are in separate syringes. The liquid components are added to the particle-loaded syringe by passing through the stopcock. After allowing the liquid carrier to adequately wet the mineralized collagen particles, they can be further mixed by passing the paste back and forth between the two syringes until a flowable composition is formed.

The embodiment can also be prepared and packaged in sterile conditions for later use. These embodiments would of course not include cellular material that can expire over time. The preferred embodiment would comprise the mineralized collagen particles and the liquid carrier containing osteogenic cytokines or gene constructs.

The matrix according to the present invention will eventually biodegrade or be absorbed, so the porosity and physical integrity cannot be maintained beyond that limiting period. This process normally takes on average about 2 to 12 weeks, and is dependent upon the size of the matrix that is implanted. However, as long as the period after which there has been complete absorption or biodegradation of the matrix has not occurred prior to the bone replacement or augmentation process, the rate of biodegradation with be sufficient.

### EXAMPLE 1

### Roto-Milled Healos

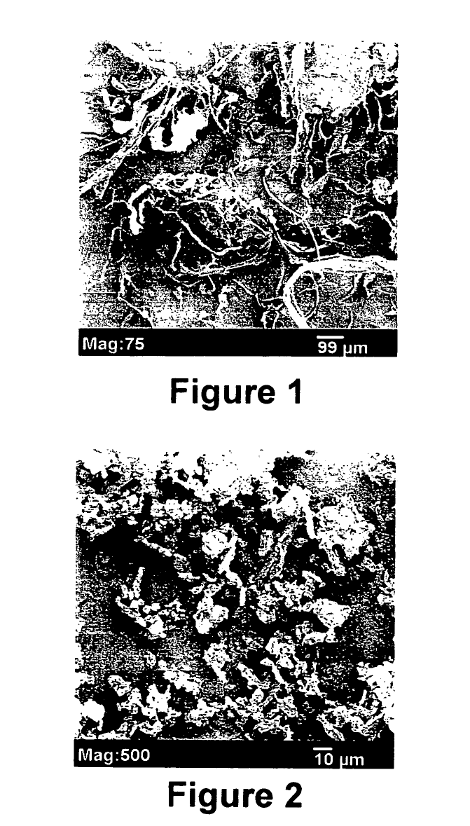

A porous, three-dimensional bone graft formed from mineralized collagen fibrils, sold under the tradename HEALOS (DePuy Spine, Inc., Mountain View, Calif.), was cut to smaller pieces of roughly 0.5 cm×2 cm and the pieces fed into a rotor-mill (Wiley Mini-Mill model 3383-L10, Thomas Scientific, Swedesboro, N.J.) fitted with a US Std. #20 mesh (Thomas Scientific, Swedesboro, N.J.). FIG. 1 shows a scanning electron micrograph of the roto-milled, irregularly shaped mineralized collagen particles comprising bound mineralized collagen fibrils.

### EXAMPLE 2

### Cryogenically-Milled Mineralized Collagen Fibrils

Mineralized collagen fibrils as described in Constantz were cut into smaller pieces and micronized with a 6800 Freezer Mill (SPEX CertiPrep, Metuchen, N.J.). The freeze/mill cycle consisted of a 20-minute initial cooling period followed by 10 cycles of 2 minutes milling and 2 minutes cooling between each milling cycle with an impact setting of 12. FIG. 2 shows a scanning electron micrograph of cryo-milled mineralized collagen fibrils.

### EXAMPLE 3

### Cryo-Milled Mineralized Collagen Fibrils Bound with Denatured Collagen

One gram of water-soluble collagen was added into 10 ml of deionized water. The solution was heated to 80° C. until the collagen was totally dissolved. The solution was cooled to and maintained at 40° C. The pH of the solution was adjusted to 7.4 using 1N NaOH. 150 milligrams of cryo-milled, mineralized collagen fibrils as show in FIG. 2 were suspended in 3 ml of the denatured collagen solution and vortexed. 3 ml of the slurry so formed was poured into 60 ml of olive oil maintained at 40° C. under stirring at 400 rpm to form agglomerates of fibrils coated with denatured collagen. Stirring was continued for 10 minutes. The solution was transferred into an ice bath and stirred for 15 minutes at 200 rpm. 400 ml of cold acetone was added into the solution while the solution still was in the ice bath. The solution was kept in the ice bath for 40 minutes. Coated agglomerates were collected by filtration. Coated agglomerates were then resuspended in 30 ml acetone. 60 micrograms of glutaraldehyde (50%) were added to the suspended coated agglomerates for crosslinking, thus forming crosslinked particles containing mineralized collagen fibrils distributed and bound there through, and the mixture incubated for 3 hours at room temperature. Crosslinked particles were washed with acetone three times. The particles were vacuum dried at room temperature.

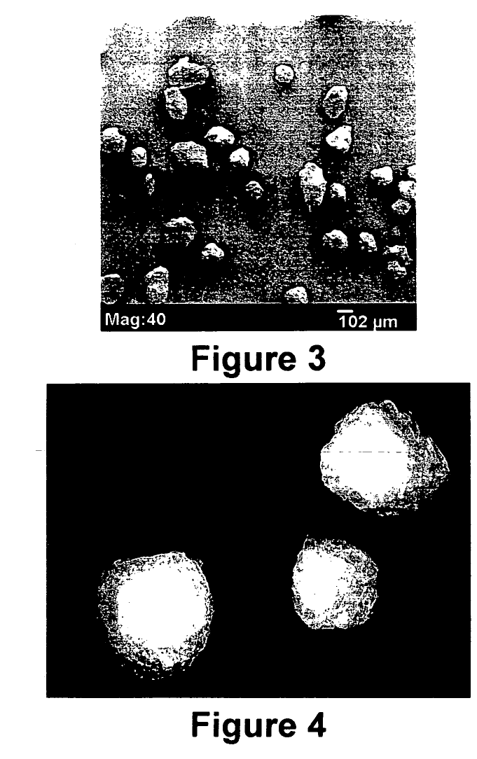

FIG. 3 shows a scanning electron micrograph of mineralized collagen particles including cryo-milled mineralized fibril agglomerates bound by denatured collagen, as prepared above. As shown, particles range in average diameter from about 100 to about 200 microns, with an aspect ratio of from about 3:1 to about 1:1.

### EXAMPLE 4

### Mineralized Collagen Fibrils Bound with Native Collagen

Water-soluble collagen and intact mineralized collagen fibrils described in Constantz were mixed in a weight ratio of 1:9. The concentration of the mixture was adjusted to 2.5% by weight by adding DI water. The pH of the slurry was adjusted to 11.12 by addition of 1N NaOH solution to obtain a homogeneous slurry. 30 ml of the aqueous slurry was then dispensed drop-wise into a stirred bath of olive oil (300 ml) maintained at a temperature between 10° C. to 30° C. After the water-in-oil emulsion formed, the particles were stabilized by the addition of 5 ml of the surfactant Span 85. After one hour of mixing, the stabilized particles were crosslinked by the addition of 0.1 ml of concentrated (27% vol/vol) glutaraldehyde solution. After one hour of stirring, the crosslinked particles, which were denser than water, were separated from the water-oil emulsion by the addition of excess water. The particles were then lyophilized by pre-freezing at −80° C. followed by holding at a vacuum of ˜0 mtorr at room temperature for approximately 24 hours.

FIG. 4 shows a micrograph of the mineralized collagen particles prepared above. As shown, such particles may be on the order of 1 to 3 millimeters. The lyophilization of the particles results in a porous structure upon vacuum removal of the frozen water from the frozen particle. The mineral content of the particles was determined to be 13%, indicating that the mineralized collagen fibrils were incorporated into the particles.

Alternatively, the particles may be prepared by the process described above using denatured collagen, i.e. gelatin, instead of native soluble collagen.

### EXAMPLE 5

### Injectability Test

Several syringes (3 ml volume) were loaded with 100 milligrams each of the particles prepared in Example 1. 1 ml of either phosphate buffered saline (PBS) or human bone marrow (HBM) liquid carrier was loaded into each syringe. The liquid carrier was mixed with the particles in each syringe by transferring the carrier into the particle-loaded syringe through a 3-way stopcock. The resulting material was injected through a 14-gauge needle and qualitatively evaluated for material integrity, including its propensity to phase-separate, and injectability. Phase separation was characterized as a disruption between the dispersed particles and the carrier in the composition, which was evident by a filtration effect within the syringe. Injectability was defined as the ease of passing the composition through the 14-gauge needle. Composition integrity was characterized based on the consistency of the composition after injection, which includes its ability to maintain its form and resist from flowing on its own accord once administered. It is noted that phase separation is a factor of material integrity in that phase separation, where a significant portion of the particles may be separated from the carrier, may lead to a less viscous composition, thus leading to undesired flow. Results are shown in Table 1.

TABLE 1

| | Phase | | |
|---|---|---|---|
| Carrier | Separation | Injectability | Integrity |
| PBS | +++ | +++ | − |
| HBM | + | ++ | + |

Integrity of the materials for purposes of injection was considered to be minimal, in that phase separation in compositions using the respective carriers was observed, as a significant portion of particles remained within the syringe after injection. Injectability for both was considered to be good, as normal force was required for injection.

Additional samples containing concentrations of 15 and 20 weight percent particles in HBM were prepared to improve properties related to injection of the compositions. Results are shown in Table 2.

TABLE 2

| | Phase | | |
|---|---|---|---|
| Concentration in HBM | Separation | Injectability | Integrity |
| 10% | + | ++ | + |
| 15% | − | + | +++ |
| 20% | − | + | +++ |

No phase separation was observed with higher concentrations of particles. The composition was thicker and hence its integrity was improved, while injection required slightly more force, although acceptable.

Injectability was further improved using carriers with viscosities higher than either PBS or HBM. Higher viscosity carriers were expected to enhance the wetability of the particles. Several syringes (3 ml volume) were loaded with 100 milligrams (10 percent by total weight of composition) each of the rotor-milled particles of Example 1. Into one syringe was loaded 1-ml of a 1% sodium hyaluronate (HA) solution (1 ml HA in 100 ml 0.9% saline solution). Into the other was loaded 1-ml of a 50:50 (w:w) blend of 1% HA solution (1 ml HA in 100 ml 0.9% saline solution) and HBM. Results are listed in Table 3. As indicated from the data, injectability of the compositions containing 10 percent by weight of particles was improved utilizing the more viscous carriers, while integrity was good, as phase separation appeared not to be an issue.

TABLE 3

| | Phase | | |
|---|---|---|---|
| Vehicle | Separation | Injectability | Integrity |
| 1% Sodium Hyaluronate | − | ++ | ++ |
| (HA) | | | |
| HA/HBM (50:50) | − | ++ | ++ |

It is noted that the data in Tables 1-3 are indicative of properties relating to compositions contemplated for administration via injection through a needle, e.g. a 14-gauge needle. Any limitations inferred do not necessarily apply to compositions that are to be administered via larger size cannula of medical devices described herein. The above description, including examples, is intended to describe certain embodiments of the current inventions and should not be used to narrow the scope of the invention, which is set forth in the appended claims.

We claim:

1. A bone graft composition suitable for administration to the body via a cannula, comprising:
mineralized collagen particles comprising bound mineralized collagen fibrils substantially uniformly distributed there through and a binder for said fibrils; and
a fluid biocompatible carrier comprising said mineralized collagen particles substantially uniformly distributed there through.

2. The composition of claim 1 wherein said carrier is selected from the group consisting of hyaluronic acid, succinalyted collagen, carboxymethyl cellulose, gelatin, collagen gel, fibrinogen, thrombin, liquid alkyd polyesters, liquid polyhydroxy compounds and bone marrow.

3. The composition of claim 1 comprising a bioactive agent.

4. The composition of claim 3 wherein the bioactive agent is selected from the group consisting of bone marrow, osteogenic growth factors, genes-encoding osteogenic growth factors, cell attachment mediators integrin-binding sequence, ligands, bone morphogenic proteins, epidermal growth factor, IGF-I, IGF-II, TGF-β I-III, growth differentiation factor, parathyroid hormone, vascular endothelial growth factor, lycoprotein, lipoprotein, bFGF, TGF-β superfamily factors, BMP-2, BMP-4, BMP-6, BMP-12, BMP-14, MP-52, sonic hedgehog, GDF5, GDF6, GDF8, PDGF, tenascin-C, fibronectin, thromboelastin, thrombin-derived peptides, heparin-binding domains, demineralized bone matrix (DBM), platelet rich plasma, bone marrow aspirate, bone fragments, bone marrow cells, mesenchymal cells, stromal cells, stem cells, embryonic stem cells, osteoblasts, precursor cells derived from adipose tissue, bone marrow-derived progenitor cells, peripheral blood progenitor cells, stem cells isolated from adult tissue and genetically transformed cells.

5. The composition of claim 3 wherein said carrier and said bioactive agent are the same.

6. The composition of claim 4 wherein said composition comprises from about 10 to about 35 weight percent of said mineralized collagen particles.

7. The composition of claim 6 wherein said mineralized collagen particles have an average diameter of from about 10 microns to about 1,000 millimeters.

8. The composition of claim 8 wherein said bioactive agent comprises human bone marrow.

9. The composition of claim 8 wherein said carrier comprises sodium hyaluronate.

10. The composition of claim 1 wherein said mineralized collagen particles have an average diameter of from about 10 microns to about 5 millimeters.

11. The composition of claim 1 comprising from about 1.5 to about 35 weight percent of said mineralized collagen particles.

12. The composition of claim 1 comprising from about 1.5 to about 7.5 weight percent of said mineralized collagen particles.

13. The composition of claim 12 wherein said mineralized collagen particles have an average diameter of from about 250 microns to about 5 millimeters.

14. The composition of claim 1 wherein said mineralized collagen particles are porous.

15. A method for the preparation of mineralized collagen particles, comprising:
preparing an aqueous solution of a water-soluble material suitable for use as a binder for mineralized collagen fibrils,
combining said mineralized collagen fibrils with said solution under conditions effective to prepare a homogenous aqueous slurry comprising said fibrils and said solution,
combining said slurry with an oil phase to form an emulsion of said slurry in said oil phase,
mixing said emulsion to form mineralized collagen particles comprising said binder material,
crosslinking said mineralized collagen particles comprising said binder material; and
isolating said crosslinked mineralized collagen particles from said emulsion, whereby said mineralized collagen particle comprises said mineralized collagen fibrils bound and substantially uniformly distributed there through.

16. The method of claim 15 wherein said water-soluble material comprises native collagen or denatured collagen.

17. The method of claim 16 wherein said water-soluble material comprises native collagen, said slurry is adjusted to a pH within about 9 to about 13 and a surfactant is added to said emulsion prior to crosslinking said mineralized collagen particles.

18. The method of claim 16 wherein said water-soluble material comprises denatured collagen.

19. The method of claim 14 wherein said mineralized collagen fibrils are micronized prior to combining with said solution of said water-soluble material.

20. The method of claim 19 wherein the average diameter of said isolated particles is from about 10 microns to about 1,000 microns.

21. The method of claim 15 wherein the average diameter of said isolated particles is from about 10 microns up to about 5 millimeters.

22. The method of claim 15 wherein said isolated particles are vacuum dried.

23. The method of claim 15 wherein said isolated particles are dried by lyophilization.
